(19) United States
(12) Patent Application Publication
(10) Pub. No.: US 2004/0138512 A1
(43) Pub. Date: Jul. 15, 2004

(54) Method for the production of propene

(76) Inventors: Michael Roper, Wachenheim (DE); Jurgen Stephan, Mannheim (DE); Gotz-Peter Schindler, Mannheim (DE)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/474,343

(22) PCT Filed: Apr. 6, 2002
(86) PCT No.: PCT/EP02/03828

(30) Foreign Application Priority Data
Apr. 12, 2001 (DE) 10118634.7
Jun. 27, 2001 (DE) DE10130958.9
Dec. 11, 2001 (DE) 10160726.1

(51) Int. Cl. C07C 6/00

(52) U.S. Cl. 585/643

(57) ABSTRACT

The invention relates to a method for the production of propene from a mixture (M1) essentially consisting of the following: ethylene (component E); hexenes (components H); optionally olefinic hydrocarbons (components K1a) different from ethylene and hexenes and optionally other inert hydrocarbons (components K1b). According to said method, the mixture (M1) is brought into contact with a metathesis catalyst at a temperature of 20 to 350° C., with the proviso that, as regards mixture (M1): the molar proportion of the sum consisting of 2-hexene and 3-hexene in components H is at least 4:1 to 99:1; the molar ratio between component E and the sum of components H and K1a is 1:1 to 100:1; the ratio between 2-hexenes and 3-hexenes is at least 2:1 as long as the 3-hexenes contained in the mixture are not simultaneously subjected to isomerization by means of which the proportion of 2-hexenes is correspondingly increased.

The present invention relates to a process for preparing propene from a mixture M1 consisting essentially of

ethylene (component E),

hexenes (components H),

if desired, olefinic hydrocarbons other than ethylene and hexenes (components K1a) and

if desired, further inert hydrocarbons (components K1b)

by bringing the mixture M1 into contact with a metathesis catalyst at from 20 to 350° C., with the following provisos in respect of mixture M1:

the mole fraction of the sum of 2- and 3-hexene in the components H is from 4:1 to 99:1

the molar ratio of component E to the sum of the components H and K1a is from 1:1 to 100:1 and

the ratio of 2-hexene to 3-hexene is at least 2:1 if the 3-hexene present in the mixture is not simultaneously subjected to an isomerization by means of which the proportion of 2-hexene is appropriately increased.

It is generally known that steam crackers operated using naphtha are predominantly used for providing unsaturated hydrocarbons which can serve as starting materials for the preparation of further value-added organic compounds. Particularly valuable starting materials are ethylene, propylene, butenes and hydrocarbons in which a benzene ring is present. Since, firstly, the spectrum of products from a steam cracker can be influenced only within narrow limits as far as the abovementioned products of value are concerned and, secondly, the demand for the individual products of value is sometimes very different, there is particular interest in devising processes for converting individual products among these products of value which may be required to a lesser extent than others in a local region or at a particular time into one another so as to be able to react flexibly to the prevailing demand for the individual products of value.

A frequent problem is that ethylene and butenes are available in sufficient quantities but propene is particularly sought after. Processes for preparing propene from other olefinic hydrocarbons by means of metathesis reactions are known, for example, from the following documents.

From U.S. Pat. No. 3,785,957 by reaction of 1-butene with 2-butene over MoO₃ and CoO on Al₂O₃.

From EP-A-0304515 by reaction of 1-butene with 2-butene over Re₂O₇/Al₂O₃.

From DE-A-19813720 by 2-stage metathesis of 1-butene with 2-butene to form propene and 2-pentene and subsequent reaction of the 2-pentene with ethylene to give propene and 1-butene.

U.S. Pat. No. 3,785,957 relates to the preparation of gasoline having a high octane number from a gasoline in which olefins are present, ethylene and isobutane. Here, the olefin-containing gasoline is disproportionated with ethylene to give a mixture comprising propene, butenes and a gasoline fraction consisting of C5 or higher hydrocarbons. This mixture is fractionated to give ethylene, propene, butenes, a fraction comprising olefinic C5- or C5-C6-hydrocarbons and a gasoline fraction comprising C6 and higher or C7 and higher hydrocarbons. The fraction comprising C5- or C5-C6-hydrocarbons is reacted with ethylene in a second disproportionation reaction to give a mixture comprising ethylene, propylene and butenes. The butenes removed from this are alkylated by means of isobutane to produce an alkylation product having a high octane content.

DE-A-10013253, which is not a prior publication, relates to a process for converting olefinic C4-hydrocarbons into propene and hexene.

The preparation of propene from hexenes becomes a particular issue when mixtures comprising 1- and 2-butene are employed for preparing propene. Although the cross-metathesis of 1-butene with 2-butene forms the desired propene, reaction of at least part of the 1-butene in a self-metathesis reaction to form hexenes is unavoidable.

It is an object of the present invention to provide a process by means of which propene can be prepared in a targeted manner from hydrocarbon fractions having a high proportion of 2- or 3-hexene. We have found that this object is achieved by a process as defined at the outset. The mixture used according to the present invention (M1) consists essentially of

ethylene (component E),

hexenes (components H),

if desired, olefinic hydrocarbons other than ethylene and hexenes (components K1a) and

if desired, further inert hydrocarbons (components K1b).

For the purposes of the present invention, hexenes (components H) are 1-, 2- and 3-hexene.

Possible olefinic hydrocarbons other than ethylene and hexenes (components K1a) are, in particular, pentenes, butenes, methylbutenes or methylpentene.

Possible further inert hydrocarbons (components K1b) are, in particular, saturated hydrocarbons such as ethane, propane, butane, isobutane, neopentane, isopentane, methylcyclopropane..

The molar ratio of the sum of 2- and 3-hexene to the remainder of the components H is preferably 10:1 and particularly preferably 100:1.

The molar ratio of component E to the sum of the components H and K1a is preferably 15:1 and particularly preferably 20:1.

The ratio of 2-hexene to 3-hexene can be chosen freely if the metathesis reaction is carried out under conditions under which isomerization of 3-hexene to 2-hexene takes place simultaneously. If it is not the case, the ratio of 2- to 3-hexene is preferably 2.5:1 and particularly preferably 3:1.

The molar ratio of the sum of the components E, H and K1a to the components K1b is preferably 4:1 and particularly preferably 10:1 and very particularly preferably 100:1.

As metathesis catalysts with which the mixture M1 is brought into contact for the desired reaction when the ratio of 2- to 3-hexene is at least 2:1, it is possible to use catalysts comprising a compound of a metal of group VIb or VIIb of the Periodic Table of the Elements. The metathesis catalyst preferably comprises an oxide of a metal of group VIb or VIIb of the Periodic Table. In particular, the metathesis catalyst is selected from the group consisting of Re₂O₇, WO₃ and MoO₃. Suitable catalysts of this type and their preparation are described, for example, in DE-A-11013253.

The reaction can be carried out either in the liquid phase or in the gas phase.

In the liquid phase, the metathesis is preferably carried out at from 0 to 110° C., while in the gas phase it is preferably carried out at from 150 to 350° C.

The pressure is generally from 10 to 15 bar if the reaction is carried out in the liquid phase and from 1 to 5 bar if the reaction is carried out in the gas phase.

Reaction times of from 1 to 5 hours are usually sufficient.

If the metathesis is to be carried out under isomerizing conditions, 2 possible methods are available:

1. The metathesis is carried out at from 110 to 350° C. and pressures of from 1 to 60 bar, particularly preferably at about 150C.° and about 5 bar, using Re₂O₇ on Al₂O₃ as catalyst.

2. Use is made of catalyst packing which comprises both the abovementioned metathesis catalysts and isomerization catalysts different therefrom. The isomerization catalysts comprise a metal of group Ia, IIa, IIIb, IVb, Vb or VIII of the Periodic Table of the Elements or a compound thereof. The isomerization catalyst is preferably selected from the group consisting of RuO₂, MgO and K₂CO₃. The catalysts are generally supported on the customary materials known to those skilled in the art. Examples of suitable materials include SiO₂, gamma-Al₂O₃, MgO or mixtures of these materials.

The reaction according to the present invention can be carried out batchwise or continuously, e.g. by continuously feeding a liquid or gas stream comprising the mixture M1 into a reaction zone, bringing it into contact with the metathesis catalyst there and continuously taking a stream 2 from the reaction zone.

The stream 2 consists essentially of

from 1 to 50 mol % of ethylene (component E)

from 1 to 30 mol % of propene (component Pr)

from 0 to 10 mol % of butenes (components B)

from 0 to 10 mol % of pentenes (components Pe)

from 15 to 40 mol % of 2-hexene (component H2)

from 0 to 25 mol % of hexenes other than 2-hexene (components Hx)

from 0 to 5 mol % of further olefinic hydrocarbons (components K1a) and

from 0 to 25 mol % of further saturated hydrocarbons (components K1b).

Possible components K1a are, for example, octenes, nonenes or decenes.

In general, the stream 2 is fractionated to give, as separate fractions, the components E, Pr, B and Pe, a mixture of the components H2 and Hx and a mixture of the other components.

The required mixtures M1 are generally obtained by subjecting a hydrocarbon fraction (mixture M2) consisting essentially of 2- or 3-hexene to an isomerization to convert 3-hexene into 2-hexene if the molar ratio of 2-hexene to 3-hexene is less than 2:1 and adding component E to the reaction mixture in amounts which conform to the definition. It is likewise possible to carry out the isomerization on the mixture M1.

Catalysts suitable for this purpose are, in particular, those which have been mentioned above in the case of the variant 2 of the isomerizing metathesis reaction as being added to the metathesis catalysts to effect isomerization of 3-hexene to 2-hexene (isomerization catalysts).

The mixture M2 is obtained particularly advantageously by bringing a mixture M3 consisting essentially of

2-butene (component B2),

1-butene (component B1),

if desired, ethylene (component E),

if desired, further olefinic hydrocarbons (components K3a) and

if desired, inert hydrocarbons (components K3b)

into contact with a metathesis catalyst at from 0 to 350° C. and separating a hydrocarbon fraction consisting essentially of 2- or 3-hexene from the reaction mixture. The reaction of such mixtures M3 is described in detail in DE-A-10013253.

The components B obtained from the stream 2 are preferably used as component in mixture M3.

The components E, H2 and Hx are preferably recirculated, i.e. used for preparing mixture M1.

The mixture M3 is preferably prepared by

subjecting naphtha or other hydrocarbon compounds to a steam cracking or FCC process (fluid catalytic cracking process) and separating off a C4-hydrocarbon fraction from the product stream,

preparing a C4-hydrocarbon stream (raffinate I) consisting essentially of isobutene, 1-butene, 2-butene and butanes from the C4-hydrocarbon fraction by hydrogenating the butadienes and butynes to butenes or butanes by means of selective hydrogenation or removing the butadienes and butynes by extractive distillation,

separating off the major part of the isobutene from the raffinate I by chemical, physicochemical or physical methods (cf., in particular, the BASF isobutene process which is described in EP-A 0 003 305 and EP-A 0 015 513) to give a raffinate II,

freeing the raffinate II of catalyst poisons by treatment with adsorbent materials to give the mixture M3.

Details of the procedure in these steps are generally known and can likewise be found in DE-A-10013253.

The mixture M3 can also, in particular, be prepared by firstly preparing a C₄-olefin mixture by one of the methods described below and treating this mixture further by methods analogous to those for raffinate I.

The C₄-olefin mixtures are generally prepared from LPG, LNG or MTO streams. The abbreviation LPG stands for liquefied petroleum gas. Liquefied gases of this type are defined, for example, in DIN 51 622. They generally comprise the hydrocarbons propane, propene, butane, butene and mixtures thereof which are obtained in oil refineries as by-products in the distillation and cracking of crude oil and in the processing of natural gas when gasoline is separated off. LNG stands for liquefied natural gas. Natural gas comprises mainly saturated hydrocarbons which, depending on their origin, have different compositions and can generally be divided into three groups. Natural gas from pure natural gas fields consists of methane and a little ethane. Natural gas from oil fields further comprises relatively large amounts of higher molecular weight hydrocarbons such as ethane, propane, isobutane, butane, hexane, heptane and by-products. Natural gas from condensate and distillate fields comprises not only methane and ethane but also considerable quantities of relatively high-boiling components having more than 7 carbon atoms. For a more detailed description of liquefied gases and natural gas, reference may be made to the corresponding keywords in Römpp, Chemielexikon, 9th edition.

The LPG and LNG used as feedstock comprises, in particular, field butenes, viz. the C4 fraction of the “wet” components of natural gas and of the gases accompanying crude oil, which are separated from the gases in liquid form by drying and cooling to about −30° C. Low-temperature or pressure distillation of these liquids gives the field butanes whose composition varies according to the field, but they generally comprise about 30% of isobutane and about 65% of n-butane.

The C₄-olefin mixtures derived from LPG or LNG streams can be obtained in an appropriate manner by isolation and dehydrogenation of the C₄ fraction and feed purification. Possible work-up sequences for LPG or LNG streams are dehydrogenation, subsequent removal or partial hydrogenation of the dienes, alkines and enines and subsequent isolation of the C₄-olefins. As an alternative, the dehydrogenation can be followed firstly by the isolation of C₄-olefins, after which the dienes, alkines and enines and, if appropriate, further by-products are separated off or partially hydrogenated. It is also possible to carry out the sequence isolation of the C₄-olefins, dehydrogenation, removal or partial hydrogenation of undesirable components.

A preferred procedure is

to prepare a C₄-olefin mixture from a hydrocarbon stream comprising butanes by dehydrogenation and subsequent isolation of the C₄-olefins

to prepare a C4-hydrocarbon stream (raffinate I) consisting essentially of isobutene, 1-butene, 2-butene and butanes from the C₄-olefin mixture by hydrogenating the butadienes and butynes to butenes or butanes by means of selective hydrogenation or removing the butadienes and butynes by extractive distillation, and

to separate off the major part of the isobutene from the raffinate I by chemical, physicochemical or physical methods to give a raffinate II.

Suitable methods of dehydrogenating hydrocarbons are described, for example, in DE-A-100 47 642. The dehydrogenation can, for example, be carried out in the presence of a heterogeneous catalyst in one or more reaction zones, with at least part of the heat of dehydrogenation required being generated directly in the reaction mixture in at least one reaction zone by combustion of hydrogen, of the hydrocarbon or hydrocarbons and/or of carbon in the presence of an oxygen-containing gas. The reaction gas mixture which comprises the dehydrogenatable hydrocarbon or hydrocarbons is brought into contact with a Lewis-acid dehydrogenation catalyst which has no Brönsted acidity. Suitable catalyst systems are Pt/Sn/Cs/K/La on oxidic supports such as ZrO₂, SiO₂, ZrO₂/SiO₂, ZrO₂/SiO₂/Al₂O₃, Al₂O₃, Mg(Al)O.

Suitable mixed oxides for the support are obtained by sequential precipitation or coprecipitation of soluble precursor substances.

Further details regarding the dehydrogenation of alkanes may be found in U.S. Pat. No. 4,788,371, WO 94/29021, U.S. Pat. No. 5,733,518, EP-A-0 838 534, WO 96/33151 or WO 96/33150.

The LNG stream can, for example, be converted into the C₄-olefin mixture by an MTO process. MTO stands for methanol-to-olefin. It is related to the MTG process (methanol-to-gasoline). It is a process for the dehydration of methanol over a suitable catalyst to give an olefinic hydrocarbon mixture. Depending on the C₁ feed stream, the MTO process can incorporate an upstream methanol synthesis. C₁ feed streams can thus be converted via methanol and the MTO process into olefin mixtures from which the C₄-olefins can be separated off by suitable methods. The separation can be carried out, for example, by distillation. For details of the MTO process, reference may be made to Weissermel, Arpe, Industrielle organische Chemie, 4th edition 1994, VCH-Verlagsgesellschaft, Weinheim, p. 36 ff.

In general, a C₄-olefin mixture is prepared from methanol by dehydration (MTO process) and a raffinate 2 is prepared from this by, if appropriate, distillation, partial hydrogenation of alkines and extractive distillation.

In addition, the methanol-to-olefin process is described, for example, in P. J. Jackson, N. White, Technologies for the conversion of natural gas, Austr. Inst. Energy Conference 1985.

In a preferred process variant, further propene is produced by cross-metathesis of 2-pentene with ethene. For this purpose, a mixture M4 is firstly prepared by adding ethene to a fraction consisting essentially of pentenes (components Pe) which has been separated off from the stream 2 in such an amount that the mixing ratio of the components E and Pe is 5:1, preferably 10:1.

The components Pe present in the mixture M4 comprise mainly 1-pentene and only minor amounts of 2-pentene. However, since 2-pentene is required for the preparation of propene, the mixture M4 is reacted under the conditions which have been described above for the isomerizing metathesis of mixture M1.

As an alternative, either the mixture M5 or the component Pe is subjected beforehand to an isomerization to achieve a ratio of 2-pentene to 1-pentene of at least 2:1. In this case, the metathesis can be carried out using catalyst packing which effects only metathesis and not any simultaneous isomerization. Here too, what has been said above in respect of the isomerization of 3- to 2-hexene applies analogously.

The cross-metathesis of ethylene and 2-pentene can be carried out batchwise or continuously, e.g. by feeding a liquid or gas stream comprising the mixture M4 continuously into a reaction zone, bringing it into contact with the metathesis catalyst there and continuously taking a stream 5 from the reaction zone.

The stream 5 consists essentially of

from 1 to 50 mol % of ethylene,

from 1 to 30 mol % of propene,

from 0 to 10 mol % of butenes,

from 0 to 10 mol % of pentenes,

from 0 to 5 mol % of further olefinic hydrocarbons (components K5a) and

from 0 to 25 mol % of further saturated hydrocarbons (components K5b).

In general, the components E, Pr, B, Pe, K5a and K5b are separated as separate fractions from the stream 5.

Unreacted components E and Pe are generally recirculated. Component E is suitable for the preparation of mixture M1 or M5, and component Pe is suitable for preparing mixture M5.

The following examples illustrate the process of the present invention:

### EXAMPLE 1

In a glove box, 40 g of catalyst (10% of Re₂O₇ on a γ-Al₂O₃ support) were placed in a 300 ml steel autoclave under a protective atmosphere. The autoclave was closed and pressurized in the cold state with 40 bar of ethylene. 180 ml of a hexene mixture having the following isomer composition:

| | |
|---|---|
| 1-hexene | 2.6% by weight |
| cis-2-hexene | 15.9% by weight |
| trans-2-hexene | 51.9% by weight |
| cis- and trans-3-hexene | 27.4% by weight |
| remainder | 2.2% by weight |

were subsequently introduced.

At 40° C., ethylene was then injected to a pressure of 200 bar and this pressure was kept constant over a reaction time of 12 hours. GC analysis indicated the following conversions:

| | Conversion of | | |
|---|---|---|---|
| | hexene | Yield of pentene | Yield of propene |
| Experiment | [%] | [% by weight] | [%]* |
| 1 | 74 | 36 | 26.5 |
| 2 | 43 | 21 | 21.4 |
| 3 | 81 | 69 | 61.2 |
| 4 | 82 | 69 | 68.1 |

*based on 2-hexene used

### EXAMPLE 2

In a glove box, 40 g of catalyst (10% of Re₂O₇ on a γ-Al₂O₃ support) were placed in a 300 ml steel autoclave under a protective atmosphere. The autoclave was closed and pressurized in the cold state with 40 bar of ethylene. 90 ml of an olefin mixture having the following composition:

| | |
|---|---|
| 1-pentene | 70.9% by weight |
| cis-2-hexene | 3.5% by weight |
| trans-2-hexene | 9.9% by weight |
| cis- and trans-3-hexene | 2.1% by weight |
| remainder (higher olefins) | 13.4% by weight |

were subsequently introduced.

The reaction temperature was then increased to 150° C. to effect isomerization -of the olefin mixture used. After the reaction temperature had been reached, ethylene was then injected to a pressure of 200 bar and this pressure was kept constant over a reaction time of 12 hours.

| | Conversion of | |
|---|---|---|
| | 1-pentene | Yield of propene |
| Experiment | [%] | [%]* |
| 5 | 34 | 46 |

*based on the sum of pentene + hexene used

### EXAMPLE 3

In a glove box, 40 g of catalyst (10% of Re₂O₇ on a γ-Al₂O₃ support) were placed in a 300 ml steel autoclave under a protective atmosphere. The autoclave was closed and pressurized in the cold state with 40 bar of ethylene.. 180 ml of a hexene mixture having the following isomer composition:

| | |
|---|---|
| 1-hexene | 0.1% by weight |
| cis-2-hexene | 0.2% by weight |
| trans-2-hexene | 0.1% by weight |
| cis- and trans-3-hexene | 99.6% by weight |
| remainder | 0.4% by weight |

were subsequently introduced.

The reaction temperature was then increased to 150° C. to effect isomerization of the 3-hexene used. After the reaction temperature had been reached, ethylene was then injected to a pressure of 200 bar and this pressure was kept constant over a reaction time of 12 hours.

GC analysis indicated the following conversions:

| | Conversion of | | |
|---|---|---|---|
| | hexene | Yield of pentene | Yield of propene |
| Experiment | [%] | [% by weight] | [%]* |
| 6 | 79 | 39 | 43 |

*based on 3-hexene used

1. A process for preparing propene from a mixture M1 consisting essentially of
ethylene (component E),
hexenes (components H),
if desired, olefinic hydrocarbons other than ethylene and hexenes (components K1a) and
if desired, further inert hydrocarbons (components K1b)
by bringing the mixture M1 into contact with a metathesis catalyst at from 20 to 350° C., with the following provisos in respect of mixture M1:
the mole fraction of the sum of 2- and 3-hexene in the components H is from 4:1 to 99:1
the molar ratio of component E to the sum of the components H and K1a is from 1:1 to 100:1 and
the ratio of 2-hexene to 3-hexene is at least 2:1 if the 3-hexene present in the mixture is not simultaneously subjected to an isomerization by means of which the proportion of 2-hexene is appropriately increased.

2. A process as claimed in claim 1, wherein the metathesis catalysts are compounds of a metal of transition group VIb, VIIb or VIII.

3. A process as claimed in claim 1 or 2, wherein a stream 1 comprising the mixture M1 is fed continuously into a reaction zone, brought into contact with the metathesis catalyst there and a stream 2 is continuously taken from the reaction zone.

4. A process as claimed in any of claims 1 to 3, wherein the isomerization of 3- to 2-hexene carried out simultaneously with the metathesis is effected by either
using Re₂O₇ on Al₂O₃ as metathesis catalyst and bringing the mixture M1 into contact with the catalyst at from 110 to 350° C. and pressures of from 1 to 60 bar, or
bringing the mixture M1 into contact with catalyst packing which comprises the abovementioned metathesis catalysts together with customary isomerization catalysts different therefrom.

5. A process as claimed in any of claims 3 to 5, wherein a stream 2 comprising
from 1 to 50 mol % of ethylene (component E)
from 1 to 30 mol % of propene (component Pr)
from 0 to 10 mol % of butenes (component B)
from 0 to 10 mol % of pentenes (component Pe)
from 15 to 40 mol % of 2-hexene (component H2)
from 0 to 25 mol % of hexenes other than 2-hexene (components Hx)
from 0 to 5 mol % of further olefinic hydrocarbons (components K2a) and
from 0 to 25 mol % of further saturated hydrocarbons (components K2b)
is separated into, as separate fractions, the components E, Pr, B, Pe, a mixture of the components H2 and Hx and a mixture of the other components.

6. A process as claimed in claim 5, wherein the components E, B, H2 and Hx are recirculated to the reaction zone.

7. A process as claimed in any of claims 1 to 6, wherein the mixture M1 is prepared by subjecting a hydrocarbon fraction consisting essentially of 2- or 3-hexene to an isomerization to convert 3-hexene into 2-hexene if the ratio of 2-hexene to 3-hexene is less than 2:1 so as to achieve the abovementioned molar ratio and, either before or after the isomerization, adding component E to the hydrocarbon fraction in such an amount that the molar ratio defined above is achieved.

8. A process as claimed in any of claims 1 to 6, wherein the hydrocarbon fraction consisting essentially of 2- or 3-hexene is prepared by bringing a mixture M3 consisting essentially of
2-butene (component B2),
1-butene (component B1),
if desired, ethylene,
if desired, further olefinic hydrocarbons (components K3a) and
if desired, inert hydrocarbons (components K3b)
into contact with a metathesis catalyst at from 0 to 350° C. and separating a hydrocarbon fraction consisting essentially of 2- or 3-hexene from the reaction mixture.

9. A process as claimed in any of claims 5 to 8, wherein the components B isolated from the stream 2 are used as a component in mixture M3.

10. A process as claimed in any of claims 1 to 8, wherein
naphtha or other hydrocarbon compounds is/are subjected to a steam cracking or FCC process and a C4-hydrocarbon fraction is separated off from the product stream,
a C4-hydrocarbon stream (raffinate I) consisting essentially of isobutene, 1-butene, 2-butene and butanes is prepared from the C4-hydrocarbon fraction by hydrogenating the butadienes and butynes to butenes or butanes by means of selective hydrogenation or removing the butadienes and butynes by extractive distillation,
separating off the major part of the isobutene from the raffinate I by chemical, physicochemical or physical methods to give a raffinate II, and
freeing the raffinate II of catalyst poisons by treatment with adsorbent materials to give the mixture M3.

11. A process as claimed in claim 8, wherein a mixture M3 is prepared by
preparing a C₄-olefin mixture from a hydrocarbon stream comprising butanes by dehydrogenation and subsequent isolation of the C₄-olefins,
preparing a C4-hydrocarbon stream (raffinate I) consisting essentially of isobutene, 1-butene, 2-butene and butanes from the C₄-olefin mixture by hydrogenating the butadienes and butynes to butenes or butanes by means of selective hydrogenation or removing the butadienes and butynes by extractive distillation, and
separating off the major part of the isobutene from the raffinate I by chemical, physicochemical or physical methods to give a raffinate II.

12. A process as claimed in claim 8, wherein a C₄-olefin mixture is prepared from methanol by dehydration (MTO process) and a raffinate 2 is prepared from this by, if appropriate, distillation, partial hydrogenation of alkines and extractive distillation.

13. A process as claimed in any of claims 5 to 12 for preparing propene using the component Pe originating from stream 2, wherein a mixture M5 consisting essentially of
components E and Pe,
if desired, olefinic hydrocarbons different from the components E and Pe (components K5a) and
if desired, further inert hydrocarbons (components K5b)
is brought into contact with a metathesis catalyst at from 20 to 350° C., with the following provisos:
the molar ratio of component E to the sum of the components Pe and K5a in the mixture M5 is from 1:1 to 100:1 and
the ratio of 2-pentene to 1-pentene is adjusted by prior isomerization of the mixture M5 or the component Pe to a ratio of at least 2:1 if the mixture M5 is not is simultaneously subjected to an isomerization by means of which the proportion of 2-pentene is increased appropriately.
